(19) United States
(12) Patent Application Publication
(10) Pub. No.: US 2004/0048313 A1
(43) Pub. Date: Mar. 11, 2004

(54) Neutrophil imaging in cystic fibrosis

(75) Inventors: Bruce Sodee, Kirtland Hills, OH (US); Michael Konstan, Bratenahl, OH (US); David M. Goldenberg, Mendham, NJ (US)

Correspondence Address: FOLEY AND LARDNER, SUITE 500, 3000 K STREET NW, WASHINGTON, DC, 20007, US

(73) Assignee: IMMUNOMEDICS, INC.

(21) Appl. No.: 10/452,917

(22) Filed: Jun. 3, 2003

Related U.S. Application Data
(60) Provisional application No. 60/386,412, filed on Jun. 7, 2002.

(51) Int. Cl. G01N 33/53

(52) U.S. Cl. 435/7.1

(57) ABSTRACT

The present invention is directed to an improved method to detect and monitor a subject having cystic fibrosis (CF) by employing a murine anti-NCA 90 monoclonal antibody Fab′ fragment and ^{99m}-technetium. It is further directed to a simple, noninvasive, and effective test that can assess neutrophil delivery to the lower airways of patients with CF and other neutrophil-mediated lung diseases.

### CROSS-REFERENCE TO RELATED APPLICATIONS

This application is based on U.S. provisional patent application serial No. 60/386,412, filed Jun. 7, 2002. The entire contents of this application, including its specification, claims and drawings, are incorporated herein by reference in their entirety.

### FIELD OF THE INVENTION

The present invention is directed to an improved method to detect and monitor a subject having cystic fibrosis (CF) by employing an antibody conjugate comprising a murine anti-NCA-90 monoclonal antibody Fab′ fragment and ^{99m}Technetium. It is further directed to a simple, noninvasive, and effective test that can assess neutrophil delivery to the lower airways of patients with CF and other neutrophil-mediated lung diseases.

### BACKGROUND OF THE INVENTION

Cystic fibrosis (CF) is the most common lethal genetic disease among Caucasians. In the United States, approximately 2,500 babies are born with CF and about 30,000 children and adults are affected by CF. CF is an autosomal recessive inherited condition that is caused by an abnormal gene on chromosome seven. The disease causes the exocrine glands of afflicted individuals to produce abnormally thick mucus that blocks passageways and produces scarring and lesions. CF affects mainly the lungs and the digestive system. In the lungs, its effects are mostly devastating; it causes increasingly severe respiratory problems. In the digestive tract, CF often results in extreme difficulty in digesting nutrients from foods.

The currently accepted pathogenic scheme for the lung disease of CF begins with a defective CF gene resulting in absent or defective protein product of the gene called Cystic Fibrosis Transmembrane Conductance Regulator (CFTR), which is involved in chloride channel activity. The primary pathophysiologic effect of this defect is believed to be the alteration of the airway environment such that abnormal mucus accounts for the airway obstruction. This is proceeded by infection with organisms with the predilection for the CF airway, such as Pseudomonas aeruginosa, Staphylococcus aureus, and Haemophilus influenzae. Besides airway obstruction by viscous secretions and chronic Pseudomonas infections as major determinants in the pathogenesis of CF, inflammation has been implicated as another major contributing factor. See Konstan, M. W. et al., Infection and Inflammation in the Lung in Cystic Fibrosis, in Cystic Fibrosis, Davis, P. B. (ed.), Marcel Dekker, Inc., NY (1993). The inflammatory response to this infection is excessive and persistent. It sets the stage for a vicious cycle of airway obstruction, infection, and inflammation that ultimately leads to lung destruction. See Davis, P. B. et al. Am. J. Respir. Crit. Care Med. 154:12291256 (1996) and Konstan, M. W. et al., Pediatr. Pulmonol. 24:137-142 (1997).

CFTR may affect the processing and chemical alterations of other proteins within the cell. The mechanism of this occurrence remains unclear. However, researchers have some evidence that an altered membrane protein in CF can serve as an attachment site for Pseudomonas and perhaps, aids in explaining the enhanced susceptibility of CF patients to infection.

Tissues that produce abnormal mucus secretions in CF include the airways, bile ducts of the liver, gastrointestinal tract (GIT), ducts of the pancreas, and male urogenital tract. Normal mucus forms a gel-like barrier that plays an important role in protecting the cells lining the inside surfaces of these tissues from infection. Instead of protecting these tissues from infection, abnormal mucus in CF obstructs airways and ducts, causing tissue damage. In addition, it provides an environment for bacteria to thrive. In response, the white blood cells or neutrophils (WBCs) are recruited to the lung to battle the infection. However, these cells die and release their sticky genetic material (DNA) into the mucus. This sticky DNA, in turn, aggravates the already formed abnormal mucus, causing further airway obstruction and infection.

The inflammatory component of CF is characterized by persistent infiltration of neutrophils, which includes times of clinical stability. See Konstan, M. W. et al., Am. J Respir. Crit. Care Med. 150:448-454 (1994). This occurs very early in the course of the disease for many patients, frequently during the first year of life, and may exist even in the absence of apparent infection. See Konstan, M. W. et al., Pediatr. Pulmonol. 24:137-142 (1997). In fact, bronchioalveolar lavage (BAL) studies done in the United States and Australia have found that even infants without the symptomatic lung disease developed significant endobronchial bacterial infections associated with inflammation and large numbers of neutrophils. See Khan, T. Z. et al., Am. J. Respir. Case Med. 151:1075-1082 (1995) and Armstrong, D. S. et al., BMJ 310:571-1572 (1995). In addition, inflammation was found to be present in some infants as early as 4 weeks of age in these two studies.

BAL studies also revealed that there is a severe local inflammatory response in CF patients with mild lung disease who appeared clinically healthy and free from pulmonary exacerbation. The airways of these patients contained a significant amount of bacteria, particularly Pseudomonas aeruginosa, and a marked increase of inflammatory cells and immunoglobulins (Igs). There was also a significant increase in the amount of uninhibited (active) neutrophil elastase in the epithelial lining fluid, presumably due to the excessive number of neutrophils in the airways. Active elastase has been shown to damage the lungs. See Bruce, M. C. et al., Am. Rev. Respir. Dis. 132:529-535 (1985). The elastase cleaves complement receptors and opsonic complement fragments from neutrophils and Pseudomonas aeruginosa, respectively, rendering opsono-phagocytosis ineffective and prolonging infection. See Berger, M. et al, J. Clin. Invest. 84:1302-1313 (1989) and Tosi, M. F. et al., J. Clin. Invest. 86:300-308 (1990).

Because of the deleterious effects of elastase and other inflammatory mediators in the CF airways, several therapeutic interventions aimed at decreasing inflammation or interfering with the injurious products of inflammation in the CF are undergoing investigation. These include anti-inflammatory agents, such as prednisone and ibuprofen (Auerbach, H. S. et al., Lancet 2:686-688 (1985) and Konstan, M. W., et al., J. Pediatr. 118:956-964 (1991)); anti-proteases such as exogenously administered α₁-PI and secretory leukoprotease inhibitor (McElvaney, N. G. et al., Lancet 337:392-394 (1991) and McElvaney, N. G. et al., J. Clin. Invest. 90:1296-1301 (1992)); and exogenously administered deoxyribonuclease (Hubbard, R. C. et al., N. Eng. J. Med. 326:812-815 (1992)). Moreover, diagnostic antibody systems are also undergoing investigation. See Gratz et al., Eur. J. Nucl. Med., 25(4): 386-93 (1998).

Regardless of how the inflammatory process is initiated and perpetuated, it has become clear that anti-inflammation therapy should be initiated early in life and that infection should be controlled to the maximum extent if possible. However, mechanistic studies of the disease are hampered by difficulties in monitoring its status which is currently done by analysis of bronchioalveolar lavage (BAL) fluid obtained via bronchoscopy. Not only does the cost and invasiveness of this procedure limit its use but the procedure also samples less than 5 percent of the lung. Neutrophil delivery to the oral mucosa can also be used as a surrogate marker for neutrophil delivery to the mucosa of the lower airways, but the assay is quite burdensome in requiring the subject to provide timed mouthwash specimens on 9 occasions over a 2-week period. Moreover, it is not known if this assay is a valid surrogate for what occurs in the lower airways of individuals with CF.

There exists in the field a continuing need to provide an early diagnostic/detection test for CF to monitor and decrease the spread of pulmonary infection in CF patients. There continues to exist a need to develop a simple effective test that can assess neutrophil delivery to the lower airways of CF patients. There further exist a need for an optimal CF assay that involves minimal risk to and require minimal input from the patient, and is less expensive than bronchoscopy with broncheoalveolar lavage, while not compromising the test's utility.

A potential viable route to this optimal test may be via radioimmunoscintography in which radiolabeled monoclonal antibodies, their fragments and related multivalent and/or multispecific constructs are exploited to target a specific biomolecule receptor which is then characterized by imaging (e.g., Hakki, S. et al., Clin. Orthopedics 335:275-285 (1997). This method has been applied to a number of human diseases and conditions, including osteomyelitis (Harwood, S. J. et al., Cell Biophysics. 24/25:99-107 (1994); Becker, W. et al., J. Nucl. Med. 35:1436-1443 (1994)), soft-tissue infection (Barron, B. et al., Surgery 125:288295 (1999)), appendicitis (Barron, B., et al., ibid.), and vasculitis (Jonker, N. D. et al., J. Nucl. Med. 33:491-497 (1992)). Despite the significant diagnostic power of these monoclonal antibodies in these areas, the applicability of this technology to assess inflammatory conditions in the lung of CF patients has not been explored. Therefore, a need exists to use radiolabeled monoclonal antibodies coupled with nuclear imaging to monitor the pulmonary inflammation in CF patients.

### SUMMARY OF THE INVENTION

Accordingly, it is one object of the present invention to provide a method for diagnosing cystic fibrosis (CF), particularly the early stages of CF thereby preventing the spread of bacterial infection and at the same time, minimizing the severity of the disease.

It is also an object of the invention to provide a safe and efficacious method to assay and monitor the extent of CF in a human subject suspected of having CF or being diagnosed for CF.

It is further an object of the invention to develop a method that can assess neutrophil/granulocyte delivery to the lower airways of CF patients.

In accomplishing this and other objects, there is provided, in accordance with one aspect of the present invention, a method for detecting and monitoring cystic fibrosis (CF) in a subject that comprises administering to the subject, an effective amount of the antibody conjugate comprising a murine anti-NCA-90 monoclonal antibody Fab′ fragment and ^{99m}-technetium, and a pharmaceutically acceptable carrier.

In accordance with another aspect of the present invention, an antibody conjugate as described above is also provided, wherein the murine antibody Fab′ fragment binds to a neutrophil epitope.

In yet another aspect of the present invention, the antibody Fab′ fragment is a MN-3 monoclonal antibody.

Other objects, features and advantages of the present invention will become apparent from the following detailed description. The detailed description and specific examples, while indicating preferred embodiments, are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples of infections where it obviously will be useful to those skilled in the prior art.

### DETAILED DESCRIPTION OF THE INVENTION

Definitions

In the description that follows, a number of terms are used extensively. The following definitions are provided to facilitate understanding of the invention.

Unless otherwise specified, “a” or “an” means “one or more.”

The term “cystic fibrosis” is defined as a lethal genetic disorder of exocrine epithelial glands of multiple organs, characterized by a wide range of symptoms including pancreatic insufficiency, dehydrated airways mucus, chronic bacterial infections of the lungs, and intestinal obstruction.

The term “anti-granulocyte antibody” refers to an antibody which recognizes an antigen which is present on one or more cell-types of the neutrophil/granulocyte/myelocyte lineage.

The term “antibody component” includes both an entire antibody and an antibody fragment.

An “antibody fragment” is a portion of an antibody such as an Fab′ fragment. Regardless of structure, an antibody fragment binds with the same antigen that is recognized by the intact antibody. For example, an anti-NCA-90 monoclonal antibody fragment binds with an epitope of NCA-90.

The term “antibody fragment” also includes any synthetic or genetically engineered protein that acts like an antibody by binding to a specific antigen to form a complex.

An antibody conjugate is a conjugate of an antibody component with a therapeutic or diagnostic agent.

Description

A potential viable route to this optimal test may be via radioimmunoscintography in which radiolabeled monoclonal antibodies, their fragments and related multivalent and/or multispecific constructs are exploited to target a specific biomolecule receptor which is then characterized by imaging (e.g., Hakki, S. et al., Clin. Orthopedics 335:275-285 (1997)). This method has been applied to a number of human diseases and conditions, including osteomyelitis (Harwood, S. J. et al., Cell Biophysics. 24/25:99-107 (1994); Becker, W. et al., J. Nucl. Med. 35:1436-1443 (1994)), soft-tissue infection (Barron, B. et al., Surgery 125:288-295 (1999)), appendicitis (Barron, B., et al., ibid.), and vasculitis (Jonker, N. D. et al., J. Nucl. Med. 33:491-497 (1992)). Despite the significant diagnostic power of these monoclonal antibodies in these areas, the applicability of this technology to assess inflammatory conditions in the lung of CF patients has not been explored. Therefore, a need exists to use radiolabeled monoclonal antibodies coupled with nuclear imaging to monitor the pulmonary inflammation in CF patients.

The inventive method utilizes ^{99m}-technetium radiolabeled murine anti-NCA-90 monoclonal antibody Fab′ fragment that binds to a neutrophil epitope, thereby tracking its migration via imaging. A preferred example of such an antibody is MN-3. See Hansen et al., Cancer 71:3478-3485 (1993); Becker et al., Semin. Nucl. Med. 24(2):142-53 (1994).

Production of Antibodies

Rodent monoclonal antibodies specific for granulocytes can be obtained by methods known to those skilled in the art. See generally, Kohler and Milstein, Nature 256:495 (1975); Coligan et al. (eds.), CURRENT PROTOCOLS IN IMMUNOLOGY (John Wiley & Sons, 1991). Briefly, monoclonal antibodies can be obtained by injecting mice with a composition comprising, for example, NCA-90, verifying the presence of antibody production by removing a serum sample, removing the spleen to obtain B-lymphocytes, fusing the B-lymphocytes with myeloma cells to produce hybridomas, cloning the hybridomas, selecting positive clones which produce anti-NCA-90 antibodies, culturing the clones that produce antibodies to the antigen and isolating the antibodies from the hybridoma cultures.

Monoclonal antibodies can be isolated and purified from hybridoma cultures by a variety of well-known techniques. Such isolation techniques include affinity chromatography with Protein-A Sepharose, size-exclusion chromatography, and ion-exchange chromatography. See Coligan et al. (eds.), CURRENT PROTOCOLS IN IMMUNOLOGY (John Wiley & Sons, 1991); Baines et al., pp. 79-104, METHODS IN MOLECULAR BIOLOGY (The Humana Press, Inc., 1992).

Suitable amounts of the NCA-90 antigen, also referred to as CD66c, can be obtained using standard techniques well-known in the art. For example, NCA-90 protein can be obtained from transfected cultured cells that overproduce NCA-90. Expression vectors that comprise DNA molecules encoding NCA-90 can be constructed using the published NCA-90 nucleotide sequence. See Oikawa et al., Biochem. Biophys. Res. Commun. 146:464-460 (1987); Wilson et al., J. Exp. Med. 173:137 (1991); Wilson et al., J. Immunol. 150:5013 (1993).

The MN-3 antibody was isolated from hybridomas derived from BALB/c mice which were immunized with partially purified carcinoembryonic antigen (CEA) derived from GW39 human colon adenocarcinoma xenografts. See Hansen et al., Cancer 71:3478-3485 (1993). The MN-3 antibody is specific for the NCA-90 antigen, a homotypic adhesion molecule expressed on granulocytes, as well as normal colonic mucosa and colonic adenocarcinoma. See Becker et al., Semin. Nucl. Med. 24(2):142-53 (1994); Watt et al., Blood 78:63-74 (1991).

MAbs can be characterized by a variety of techniques that are well-known to those of skill in the art. For example, the ability of a MAb to bind to a particular antigen can be verified using an indirect immunofluorescence assay, flow cytometry analysis, or Western analysis.

Production of Antibody Fragments

The present invention contemplates the use of fragments of murine anti-NCA-90 antibody Fab′ fragment. Antibody fragments which recognize specific epitopes can be generated by known techniques. The antibody fragments are antigen binding portions of an antibody, such as Fab′. Fab′ fragments can be generated by reducing disulfide bridges of the F(ab)′₂ fragments. These methods are described, for example, by Goldenberg, U.S. Pat. Nos. 4,036,945 and 4,331,647 and references contained therein, which patents are incorporated herein in their entireties by reference. Also, see Nisonoff et al., Arch Biochem. Biophys. 89: 230 (1960); Porter, Biochem. J. 73: 119 (1959), Edelman et al., in METHODS IN ENZYMOLOGY VOL. 1, page 422 (Academic Press 1967), and Coligan at pages 2.8.12.8.10 and 2.10.-2.10.4. Alternatively, Fab′ expression libraries can be constructed (Huse et al., 1989, Science, 246:1274-1281) to allow rapid and easy identification of monoclonal Fab′ fragments with the desired specificity. The present invention encompasses antibody fragments.

The antibody with the diagnostic agent may be provided as a kit for human or mammalian diagnostic use in a pharmaceutically acceptable injection vehicle, preferably phosphate-buffered saline (PBS) at physiological pH and concentration. The preparation preferably will be sterile, especially if it is intended for use in humans. Optional components of such kits include stabilizers, buffers, labeling reagents, second antibody for enhanced clearance, and conventional syringes, columns, vials and the like.

Administration of the antibody conjugate to a patient can be intravenous, intraarterial, intraperitoneal, intramuscular, subcutaneous or intrapleural. When administering by injection, the administration may be by continuous infusion or by single or multiple boluses.

In general, the dosage of administration will vary depending upon such factors as the patient's age, weight, height, sex, general medical condition, disease state and previous medical history. Typically, it is desirable to provide the recipient with a dosage of antibody which is in the range of from about 1 pg/kg to 2 mg/kg (amount of agent/body weight of patient), although a lower or higher dosage also may be administered as circumstances dictate, such as 1 to 5 mg per total dose administered. The label selected may also affect the dosage requirements.

The embodiments of the invention may be further illustrated through the example which shows aspects of the invention in detail. This example illustrates specific elements of the invention and is not to be construed as limiting the scope of the claims.

### EXAMPLE 1

An 18-year-old man with a history of cystic fibrosis presents to his pulmonologist with a moderate lung disease and is experiencing pulmonary exacerbation of his disease. The physician is considering a course of i.v. antibiotic therapy, but decides to establish a baseline of pulmonary imaging with a radiolabeled anti-granulocyte antibody administered i.v., followed by scintigraphic imaging of the chest with a chest gamma scans, including SPECT. The patient receives 20 mCi of ^{99m}-Tc-labeled anti-granulocyte antibody MN-3 Fab′ (LeukoScan®) in a protein dose of 0.25 mg i.v. Twenty-four hours later, gamma and SPECT images of the chest display increased uptake (about 3-fold), as compared to similar studies in patients without pulmonary disease. At this time, pulmonary function tests also indicates compromise of lung function, by about 40%. The patient was given 2 weeks of i.v. antibiotic therapy, and returned for reevaluation tests ten days later. The LeukoScan® pulmonary images shows a reduction of lung inflammation, and this is associated with a 25% improvement in pulmonary function in the predicted FEV1 as compared before and after antibiotic therapy. This result shows that this non-invasive granulocyte imaging method correctly assesses CF lung disease, and a response to i.v. antibiotic therapy.

It will be apparent to those skilled in the art that various modifications and variations can be made to the products, compositions, methods. and processes of this invention. Thus, it is intended that the present invention covers such modifications and variations, provided they come within the scope of the appended claims and their equivalents.

The disclosure of all publications cited above are expressly incorporated herein by reference in their entireties to the same extent as if each were incorporated by reference individually.

We claim:

1. A method for detecting and monitoring cystic fibrosis (CF) in a subject, comprising administering to said subject, an effective amount of an antibody conjugate and a pharmaceutically acceptable carrier,
wherein said antibody conjugate comprises a murine anti-NCA-90 monoclonal antibody Fab′ fragment and ^{99m}-technetium and wherein said antibody Fab′ fragment binds to a neutrophil epitope.

2. The method of claim 1, wherein said antibody Fab′ fragment is MN-3 monoclonal antibody.

3. The method of claim 1, wherein said antibody conjugate is administered intravenously, intraarterially, intraperitoneally, intramuscularly, subcutaneously or intrapleurally.

4. An antibody conjugate comprising a murine anti-NCA-90 antibody Fab′ fragment and ^{99m}-technetium, wherein said antibody Fab′ fragment binds to a neutrophil epitope

5. The antibody conjugate of claim 4, wherein said antibody Fab′ fragment is MN-3 monoclonal antibody.
